(19) United States
(12) Plant Patent Application Publication
(10) Pub. No.: US 2009/0260121 P1
(43) Pub. Date: Oct. 15, 2009

(54) LOBELIA PLANT NAMED 'KLELE07246'

(50) ;

(76) Inventor: Nils Klemm, Stuttgart (DE)

Correspondence Address: JONDLE & ASSOCIATES P.C., 858 HAPPY CANYON ROAD SUITE 230, CASTLE ROCK, CO, 80108, US

(21) Appl. No.: 12/082,734

(22) Filed: Apr. 14, 2008

(51) Int. Cl. A01H 5/00

(52) U.S. Cl. PLT/451

(57) ABSTRACT

A new cultivar of Lobelia particularly characterized by brilliant blue flowers with large, white eyes, strong vigor and a trailing plant habit, is disclosed.

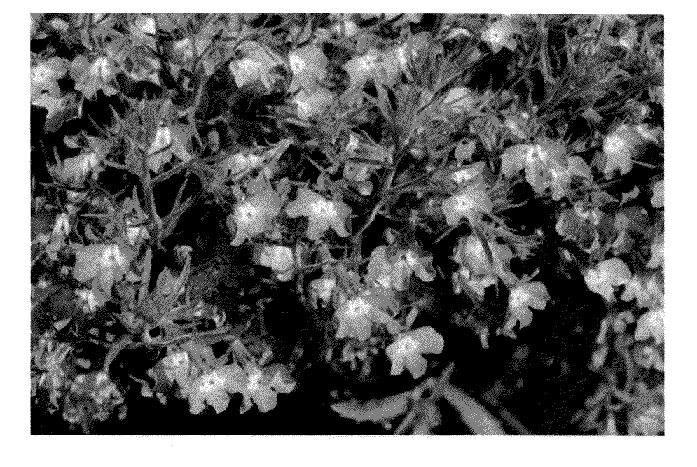

### GENUS AND SPECIES

Lobelia erinus

### VARIETY DENOMINATION

‘KLELE07246’

### BACKGROUND OF THE NEW PLANT

The present invention comprises a new and distinct cultivar of Lobelia, botanically known as Lobelia erinus, and hereinafter referred to by the cultivar name ‘KLELE07246’. ‘KLELE07246’ originated from an open pollinization made in May 2004 in Stuttgart, Germany between the proprietary female Lobelia plant ‘040312’ (unpatented) and the male plant, an unnamed individual Lobelia plant (unpatented). A single plant selection was subsequently chosen for further evaluation and for asexual propagation.

The new cultivar was created in 2004 in Stuttgart, Germany and has been asexually reproduced repeatedly by vegetative cuttings and in-vitro propagation in Stuttgart, Germany over a three-year period (about 7 generations). ‘KLELE07246’ has been found to retain its distinctive characteristics through successive asexual propagations.

### SUMMARY OF THE INVENTION

The following are the most outstanding and distinguishing characteristics of this new cultivar when grown under normal commercial practices in a greenhouse in Stuttgart, Germany.
1. Brilliant blue flowers with large, white eyes;
2. Strong vigor; and
3. A trailing plant habit.

### DESCRIPTION OF THE PHOTOGRAPH

This new Lobelia plant is illustrated by the accompanying photograph which shows a close-up of the mature flowers, buds and foliage; the colors shown are as true as can be reasonably obtained by conventional photographic procedures. The photograph is of a plant about 3-months old and grown from rooted cuttings in 10-cm pots in a greenhouse (glasshouse) in Stuttgart, Germany between February and May under conditions which approximate those generally used in commercial practice.

### DESCRIPTION OF THE NEW CULTIVAR

The following detailed descriptions set forth the distinctive characteristics of ‘KLELE07246’. The data which define these characteristics were collected from asexual reproductions carried out in Stuttgart, Germany. The plant history was taken on 2-month old plants in 10-cm pots that were pinched once and grown in a greenhouse in the spring. The color readings were determined under natural light. Color references are to the R.H.S. Colour Chart of The Royal Horticultural Society of London (R.H.S.) (2001).

### DESCRIPTION OF THE NEW PLANT

Classification:
   Family.—Campanulaceae.
   Genus and species.—Lobelia erinus.
   Common name.—Lobelia.
Parentage:
   Female parent.—The proprietary Lobelia plant ‘040312’ (unpatented).
   Male parent.—An unnamed individual Lobelia plant (unpatented).
Plant:
   Growth and branching habit.—Vigorous, moderate branching.
   Form.—Trailing.
   Height (measured from the top of the soil, including any flowers).—14 cm.
   Width (horizontal plant diameter, including any flowers).—35 cm.
   Time to produce a finished flowering plant.—9 weeks.
   Outdoor plant performance.—Good.
   Time to initiate roots.—3 weeks.
   Root description.—Fine, white, moderate branching.
Stems:
   Length.—20 cm to 25 cm.
   Diameter.—0.15 cm.
   Internode length.—2 cm to 5 cm.
   Color.—RHS 141B.
   Texture.—Slight pubescence and ribbed.
   Stem anthocyanin.—Absent.
   Peduncle.—Color: RHS 141B Length: 2 cm to 3 cm Diameter: 0.03 cm to 0.05 cm Texture: Slight pubescence.
Leaves:
   Color, both mature and immature leaves.—Upper surface: RHS 137A Lower surface: RHS 137D.
   Length.—Basal leaves and upper leaves are 3 cm to 4 cm.
   Width.—Basal leaves are about 2 cm, while upper leaves are about 1 cm.
   Shape.—Basal leaves are elliptic while upper leaves are lanceolate.
   Margin.—Serrate.
   Apex.—Obtuse.
   Base.—Acute.
   Texture (both surfaces).—Smooth.
   Venation pattern.—Pinnate.
   Venation color.—RHS 142B.
Flower bud:
   Shape.—Inverse drop.
   Diameter.—0.4 cm to 0.6 cm.
   Length.—1.0 cm to 1.2 cm.
   Color when bud is first starting to open.—Greenish-white to bluish.
Inflorescence:
   Lastingness of individual blooms on plant.—5 to 7 days.
   Fragrance.—Absent.
   Inflorescence type.—Raceme.
   Quantity of flowers and buds per flowering branch.—10 to 15.
Flowers:
   Flower diameter (horizontal).—About 2 cm.
   Flower height (vertical).—About 2 cm.
   Immature flower.—Color, upper and lower lobes: Upper surface: RHS 104B Lower surface: RHS 98D.
   Mature flower.—Upper lobe: Color: Upper surface: RHS 104B Lower surface: RHS 98D Length: 1 cm Width: 0.8 cm Shape: Obovate Apex: Obtuse Margin: Entire Texture (both surfaces): Smooth Lower lobes: Color: Upper surface: RHS 104B Lower surface: RHS 98D Length: About 0.6 cm Width: About 0.2 cm Shape: Obovate Apex: Obtuse Margin: Entire Texture (both surfaces): Smooth.
   Corolla.—Color: Inside: RHS 104B, white with blue spots Outside: White Tube length: About 0.8 cm Diameter: 0.25 cm.
   Calyx.—Shape: Funnel, star-shaped (sepals are fused in the lower half) Sepals: Color (both upper and lower surfaces): RHS 141B Length: About 1.2 cm Diameter: About 1.5 cm Apex: Acute Texture: Slight pubescence.
Reproductive organs:
   Androecium.—Number of anthers: 5 Filament length: About 0.7 cm Filament color: Bluish-white Pollen amount: Abundant.
   Gynoecium.—Pistil length: About 0.5 cm Stigma color: Dark violet Style color: Light-green Style length: About 1.0 cm.
Fruit and seed set: Seeds are small, round, 0.05 cm wide and dark black/brown in color.
Disease and insect resistance: Not observed.

### COMPARISON WITH PARENTAL AND COMMERCIAL CULTIVARS

‘KLELE07246’ differs from the female parent, the proprietary Lobelia ‘040312’ (unpatented) in that ‘KLELE07246’ has medium-green foliage, while ‘040312’ has greyish-green foliage. Additionally, the foliage of ‘KLELE07246’ is softer than ‘040312’.

‘KLELE07246’ differs from the commercial comparison variety, ‘Hot White Spot’ (patent status unknown) in that ‘KLELE07246’ has softer leaves and a greater contrast between the flower color and eye color than ‘Hot White Spot’. Additionally, ‘KLELE07246’ has medium-green foliage, while ‘Hot White Spot’ has greyish-green foliage.

I claim:

1. A new and distinct cultivar of Lobelia plant as shown and described herein.
